(19) United States
(12) Patent Application Publication
(10) Pub. No.: US 2005/0037986 A1
(43) Pub. Date: Feb. 17, 2005

(54) p53 treatment of papillomavirus and carcinogen-transformed cells in hyperplastic lesions

(76) Inventor: George H. Yoo, Grosse Pointe Park, MI (US)

Correspondence Address: FULBRIGHT & JAWORSKI L.L.P., 600 CONGRESS AVE., SUITE 2400, AUSTIN, TX, 78701, US

(21) Appl. No.: 10/747,798

(22) Filed: Dec. 29, 2003

Related U.S. Application Data
(60) Provisional application No. 60/436,754, filed on Dec. 27, 2002.

(51) Int. Cl. A61K 48/00

(52) U.S. Cl. 514/44; 424/93.2

(57) ABSTRACT

Methods for the prevention, suppression, and inhibition of growth of a papilloma-virus transformed cell in a hyperplastic lesion using a topically applied p53 expression cassette are disclosed. In addition, there are provided pharmaceutical preparations of a p53 expression cassette suitable for topical delivery to a papillomavirus-transformed cell in a hyperplastic lesion.

### BACKGROUND OF THE INVENTION

This application claims the benefit of the filing date of U.S. provisional patent application Ser. No. 60/436,754, filed Dec. 27, 2002, the entire contents of which are hereby incorporated by reference.

1. Field of the Invention

The present invention relates generally to the fields of cancer biology, molecular biology and pharmacology. More particularly, it pertains to methods and compositions for the treatment of papillomavirus- and carcinogen-transformed cells in hyperplastic lesions using p53 gene therapy. It also pertains to methods and compositions to prevent development of hyperplastic lesions composed of papillomavirus- and carcinogen-transformed cells using p53 gene therapy.

2. Description of Related Art

Lesions associated with human papillomavirus (HPV) are a major cause of morbidity and mortality in the U.S. Papillomaviruses are small DNA viruses, non-enveloped, that replicate in the nucleus of squamous epithelial cells. To date, there have been about 58 distinct HPVs identified, based on the extent and degree of relatedness of their genomes.

Many proliferative conditions are known to be associated with papillomaviruses. Examples include benign lesions such as cutaneous warts and anogenital warts and premalignant lesions such as epidermodysplasia verruciformis. Papillomaviruses are also associated with malignant lesions including carcinomas of the head and neck, cervix, anus, and penis. In 1998, the American Cancer Society estimated that 60,000 Americans would be diagnosed with head and neck cancer. HPV has been linked to 15-46% of cases and head and neck squamous cell carcinoma (HNSCC) (Steinberg and DiLorenzo, 1996). Patients with early stage HNSCC or patients who are cured from advanced cancers have a low probability of death from their primary cancer but have a significant chance of dying from a second primary tumor. More importantly, treatment (chemoprevention) of high-risk populations may reduce the development of a second primary tumor and therefore significantly improve survival (Khuri et al., 1997). Two chemoprevention trials using 13-cis-retinoic acid (CRA) have demonstrated the efficacy of clinically reversing premalignant lesions (Hong et al., 1986) and reducing the risk of secondary primary tumors (Hong et al., 1990). However, CRA is toxic, poorly tolerated and loses its preventative effects after discontinuation of therapy.

Many alterations occur during the progression to HNSCC. Indeed, many genetic alterations have been identified before histologic changes are found in the mucosa through micrometastasis (Bedi et al., 1996) or by field cancerization (Lydiatt et al., 1998). The p53 gene is a tumor suppressor gene and a transcription regulator of DNA repair, cell cycle, apoptosis, senescence, and genomic stability. The p53 gene is mutated in approximately 50% of human cancers (Boyle et al., 1993) and in 33-45% of tumors in patients with HNSCC (Koch et al., 1996). Overexpression of p53 in head and neck carcinoma cells has demonstrated tumor growth suppression using in vitro and in vivo models, in both mutated or nonmutated p53 human HNSCC cell lines (Clayman et al., 1995; Clayman et al., 1999). Injection of adenovirus-p53 (Ad-p53) into microscopic residual head and neck tumor beds of mice improved tumor control and survival rates. The efficacy of p53 gene transfer using an adenoviral vector currently is being tested in patients with HNSCC (Clayman et al., 1999; Bier-Laning et al. 1999; Clayman et al., 1998).

HPV can lead to loss of cell cycle regulation and the development of HNSCC. Recent studies have shown that HNSCC caused by HPV are of higher prevalence in oropharynx sites and have distinct biologic and clinical behaviors (Gillison et al., 2000). HPV can lead to loss of cell cycle regulation by inactivation of p53 and Rb through the E6 and E7 HPV products, respectively. E6 inactivates the p53 gene by enhanced protein degradation. The E6 and E7 products from HPV cause the inactivation of p53 and retinoblastoma (Rb) proteins. Restoration of p53 function and cell cycle regulation in patients at risk for HNSCC could potentially prevent the development of HNSCC in both carcinogen-inducedp53 mutational inactivation and HPV-E6 inhibition.

Tobacco carcinogen has also been linked to HNSCC (Schuller et al., 1990; Wei et al., 1996). Indeed, tobacco carcinogens are the primary etiologic agents involved in the genetic transformation of upper airway and digestive tract mucosa and have been linked to direct mutations of the p53 gene (Denissenko et al., 1996). Many of the effects mediated through p53 gene transfer may overcome alterations induced by tobacco carcinogenesis. In vitro transformation of immortalized human gingival keratinocytes with a tobacco carcinogen have resulted in features of carcinoma and in the activation of VEGF secretion associated with angiogenesis (Yoo et al., 2000). Expression of exogenous p53 through gene transfer has been shown to have a bystander effect through the suppression of angiogenesis (Riccioni et al., 1998; Nishizaki et al., 1999). Therefore, the dysregulation of angiogenesis in HNSCC (Sauter et al., 1999) and in these immortalized keratinocytes (Yoo et al., 2000) may be modulated through p53 gene transfer.

Treatments for advanced head and neck carcinoma include surgery, radiotherapy and/or chemotherapy. However, newer biologic therapies, such as p53 therapy, are needed. Such a therapy would be a logical strategy for preventing or inhibiting the development of HNSCC, particularly since the p53 mutation is an early genetic alteration in the development of HNSCC. This strategy can be used to prevent or inhibit the growth of other hyperproliferative lesions.

### SUMMARY OF THE INVENTION

Accordingly, one of the objects of the present invention is to provide a novel method for inhibiting the growth of a papillomavirus-transformed cell in a hyperplastic lesion in a subject by topically administering to the subject a composition comprising (a) an expression cassette comprising a promoter, active in the cells of the lesion, operably linked to a polynucleotide encoding a p53 polypeptide, and (b) a pharmaceutical preparation suitable for topical delivery, wherein expression of the p53 polypeptide inhibits growth of the cell. In preferred embodiments, the subject is a mammal or a human.

A “papillomavirus-transformed cell” is defined as a cell wherein there has been transfer of genetic information from the papillomavirus into the cell. Thus, for instance, a squamous epithelial cell containing papillomavirus genetic material in the nucleus is a papillomavirus-transformed cell. The cell can be a keratinocyte, an epithelial cell, a skin cell, a mucosal cell, or any other cell that can undergo transformation by a papillomavirus. The papillomavirus-transformed cell may express the E6 and E7 HPV products. The hyperplastic lesion can be a squamous cell hyperplastic lesion, a premalignant epithelia lesion, a psoriatic lesion, a cutaneous wart, a periungual wart, an anogenital wart, epidermodysplasi verruciformis, an intraepithelial neoplastic lesion, a focal epithelial hyperplasia, a conjunctival papilloma, a conjunctival carcinoma, a squamous carcinoma, or any pathologic change in tissue which demonstrates wherein there is an increase in the number of cells. In a specific embodiment, the papillomavirus is a human papillomavirus. In a specific embodiment, the expression cassette is carried in a viral vector. Although use of the adenoviral vector is a specific embodiment, the claimed invention contemplates use of other viral vectors such as a retroviral vector, a vaccinia viral vector, or a pox virus vector. The expression cassette can also be carried in a nonviral vector, such as a lipid or liposome.

Although any composition can be used, the composition is formulated as a mouthwash or mouthrinse in a specific embodiment. The mouthwash or mouthrinse may include a flavorant, such as wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, cinnamon leaf oil, and mixtures thereof. Other examples of compositions include a douche solution, an ointment or salve, a cream for topical, anal or vaginal deliver, a spray or aerosol, or a suppository for anal or vaginal delivery.

Examples of promoters which can be used include a constitutive promoter, an inducible promoter, or a tissue-specific promoter. Although the invention contemplates any means of growth inhibition of the hyperplastic lesion, examples of inhibiting growth include slowing or halting growth of the lesion, reduction in size of the lesion, induction of apoptosis of the lesion, or induction of an immune response against the cells of the lesion.

The claimed invention also contemplates use of other therapies against hyperplastic lesions in the same subject. For example, the subject may also receive prior, during or after therapy with the claimed invention any or all of the following: chemotherapy, radiotherapy, immunotherapy, phototherapy, cryotherpay, toxin therapy, hormonal therapy or surgery.

It is another object of the claimed invention to provide novel compositions for inhibiting the growth of a papillomavirus-transformed cell in a hyperplastic lesion in a subject. In one embodiment, the composition is a mouthwash comprising (a) an expression cassette comprising a promoter operably linked to a polynucleotide encoding a p53 polypeptide, and (b) a liquid carrier formulated for oral delivery. The mouthwash may or may not include a flavorant of the group previously described. In another embodiment, the composition is a douche solution comprising (a) an expression cassette comprising a promoter operably linked to a polynucleotide encoding a p53 polypeptide, and (b) a liquid carrier formulated for vaginal delivery. Another embodiment is a suppository containing (a) an expression cassette comprising a promoter operably linked to a polynucleotide encoding a p53 polypeptide, and (b) formulated for anal or vaginal delivery. Another embodiment is a cream comprising the same expression cassette, formulated for topical, anal, or vaginal delivery. Other embodiments include a solution formulated as a hypostray and an aerosolized suspension.

Further, it is an object of the claimed invention to provide novel methods of suppressing or preventing papillomavirus-mediated transformation of a cell in a subject comprising administering to the cell a composition comprising (a) an expression cassette comprising a promoter, active in the cell operably linked to a polynucleotide encoding a p53 polypeptide, and (b) a pharmaceutical preparation suitable for topical delivery wherein expression of the p53 polypeptide suppresses the transformation of the cell. In a certain embodiment, the cell is a keratinocyte. In a specific embodiment, the subject is a human at risk of developing an oral hyperplastic lesion. Examples of such oral hyperplastic lesions include premalignant epithelial cells, squamous intraepithelial neoplastic cells, squamous hyperplastic cells, and squamous carcinoma cells. In a specific embodiment, the oral hyperplastic lesion is comprised of cells transformed by a papillomavirus. The papillomavirus may or may not be a human papillomavirus. In a specific embodiment, the expression cassette is carried in a viral vector. Although use of an adenoviral vector is a specific embodiment, other viral vectors such as retroviral vectors adeno-associated viral vectors, vaccinia viral vectors, and pox viral vectors can be used. In other embodiments, the expression is carried in a nonviral vector. Examples of nonviral vectors that are contemplated include lipids and liposomes. In a specific embodiment, the composition is formulated as a mouthwash. The mouthwash may or may not contain a flavorant of the list previously described. Examples of other compositions include a douche solution for vaginal delivery, a suppository for anal or vaginal delivery, an ointment or salve for topical delivery, a cream for topical, anal, or vaginal delivery, and a spray or aerosol for topical delivery. The composition can also be formulated as a pill or capsule. Finally, the composition may or may not be formulated for timed-release.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.” Throughout this application, the term “about” is used to indicate that a value includes the standard deviation of error for the device or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates the percentage of IHGK, IHGKN, HN12, and HN30 cells staining with X-Gal after transfecting with Ad-βgal.

FIG. 2 illustrates proliferation (³H-thymidine incorporation, counts per minute[cpm]) inhibition induced by Ad-p53 or Ad-βgal at 24, 48 and 72 hours after transfecting (a) IHGK, (b) IHGKN, (c) HN12 and (d) HN30 cells. Viral particle to cell VPC ratios and bGAL=βGAL.

FIG. 3 illustrates G0/G1 arrest of IHGK, IHGKN, HN12, and HN30 cells after 3 days of transfection with Ad-βgal and Ad-p53 (viral particle to cell=VPC ratios). Note: VPC ratios of 100 and 500 were not performed on IHGKN, HN30, or HN12 cells because no transduction was observed at these levels. VPC: viral particle to cell.

FIG. 4 illustrates expression of p53 and p21 by Western blot analysis in IHGK, IHGKN, HN12, and HN30 cells after 48 hours transfecting with Ad-βgal and Ad-p53.

FIG. 5 illustrates apoptosis (measured as % annexin binding) measured by flow cytometry 48 hours after transfecting with Ad-βgal and Ad-p53 in IHGK, IHGKN, HN12, and HN30 cells (viral particle to cell=VPC ratios). Note: VPC ratios of 5000 and 10,000 were not performed on IHGK cells because 100% transduction rate was achieved at a VPC of 1000 and higher. VPC ratios of 100 and 500 were not performed on IHGKN, HN30, or HN12 cells because no transduction was observed at these levels. VPC: viral particle to cell.

FIG. 6 illustrates apoptosis of HN12. Annexin binding was measured between 15 and 48 hours after transfecting with Ad-βgal and Ad-p53 in HN12 cells at VPC ratios of 10,000.

### DESCRIPTION OF ILLUSTRATIVE EMBODIMENTS

Many hyperproliferative conditions are known to be associated with human papillomaviruses (HPV). Examples range from benign lesions such as cutaneous and anogenital warts to premalignant lesions such as epidermodysplasia verruciformis to malignancies. In particular, HPV has been linked to 15-46% of cases and head and neck squamous cell carcinoma (HNSCC) (Steinberg and DiLorenzo, 1996) and plays a significant role in the genesis of other cancers, such as cervical carcinoma (See, e.g. Furumoto and Irahara, 2002; Jastreboff and Cymet, 2002; Bosch et al., 2002). The p53 gene is a tumor suppressor gene and a transcription regulator of DNA repair, cell cycle, apoptosis, senescence, and genomic stability. The p53 gene is mutated in approximately 50% of human cancers (Boyle et al., 1993). The E6 and E7 products from HPV infection cause the inactivation of p53 and retinoblastoma (Rb) proteins. Thus, methods and agents are needed to restore p53 function and cell cycle regulation to prevent or inhibit the growth of hyperproliferative lesions associated with HPV.

As discussed herein, the experimental findings of the inventors demonstrate that overexpression of p53 suppresses growth in HPV-immortalized and carcinogen-transformed oral keratinocytes. HPV-immortalized gingival keratinocytes have some features that resemble preneoplastic upper airway and digestive tract cells because the transformed cells (a) are not tumorigenic in nude mice (Oda et al., 1996) and (b) form dysplastic squamous tissue on organotypic raft cultures (Yoo et al., 2000). Thus, the inventors propose that exogenous administration of the p53 gene can be used to treat HNSCC that is causally related to carcinogens or HPV. In addition, the results suggest that restoration of p53 function and cell cycle regulation in patients at risk for HNSCC can potentially prevent the development of HNSCC in both carcinogen-induced p53 mutational inactivation and HPV-E6 inhibition. Further, these same measures can have clinical application in the treatment and prevention of other hyperplastic lesions caused by HPV.

A. Papillomavirus

Many proliferative conditions are known to be associated with papillomaviruses, in particular varieties of warts, such as condyloma acuminata (anogenital warts). The clinical importance of warts varies considerably and determinative factors are the infecting viral type, the location of the wart, and factors unique to the host. For example, a wart located on the skin is often clinically insignificant, being self limiting. However, warts on the vocal cords may be life threatening as a result of respiratory obstruction. The vast majority of skin warts spontaneously regress within a few years after their initial appearance, but may persist for longer times. The exception is a rare life threatening papillomavirus disease termed epidermodysplasia verruciformis. In this disease, the infected individual does not experience spontaneous regression, but rather the infection may progress to a malignant stage (Salzman and Howley, 1987).

Papillomaviruses are also implicated in a number of cancers. Individual types of human papillomaviruses (HPV) which infect mucosal surfaces have been implicated as the causative agents for carcinomas of the cervix, anus, penis, larynx and the buccal cavity, occasional periungal carcinomas, as well as benign anogenital warts. The identification of particular HPV types is used for identifying patients with premalignant lesions who are at risk of progression to malignancy. Although visible anogenital lesions are present in some persons infected with human papillomavirus, the majority of individuals with HPV genital tract infection do not have clinically apparent disease, but analysis of cytomorphological traits present in cervical smears can be used to detect HPV infection. Conventional viral detection assays, including serologic assays and growth in cell culture, are not commercially available and/or are not suitable for the diagnosis and tracking of HPV infection. Papanicolaou tests are a valuable screening tool, but they miss a large proportion of HPV-infected persons.

HPV has been found to contribute to the genesis of cervical cancer (See, e.g. Furumoto and Irahara, 2002; Jastreboff and Cymet, 2002; Bosch et al., 2002). HPV has two transforming genes that encode the oncoproteins E6 and E7. E6 can form complexes with p53 and promote p53 degradation. Exogenous expression of p53 in HPV-infected cervical carcinoma cells through wild-typep53 gene transfer has been shown to inhibit in vitro growth and induction of apoptosis (Hamada et al., 1996).

Papillomaviruses are also involved in producing sexually transmitted warts of the genital tract. It is reported that well over a million cases exist in the United States alone (Beckter et al., 1987).

The intact DNA of human papillomavirus (HPV) is supercoiled and thus resembles an endless loop of twisted telephone handset cord. Inside this shell, the viral DNA is packaged in and around proteins from the cell nucleus, histones, and associated peptides, into a structure that resembles cellular chromatin (Turek, 1994). Human papillomaviruses characterized to date are associated with lesions confined to the epithelial layers of skin, or oral, pharyngeal, respiratory, and anogenital mucosae. Specific human papillomavirus types, including HPV 6 and 11, frequently cause benign mucosal lesions, whereas other types. HPV 16, 18, and a host of other strains, are predominantly found in high-grade lesions and cancer. All human and animal papillomaviruses appear to share a similar genetic organization, although there are differences in the functions of individual viral genes and in their regulation. The most common genital HPV type associated with cervical carcinoma, HPV 16, has been studied most extensively.

All large open reading frames (ORFs) in HPV are on one DNA strand. Papillomaviral mRNAs appear to be transcribed solely from a single strand in infected cells. The viral genome can be divided into three regions, the upstream regulatory region (URR), or long control region (LCR), containing control sequences for HPV replication and gene expression, the viral early gene region, encoding, among others, the E2, E6 and E7 genes, and the late region, encoding the L1 and L2 genes. (Turek, 1994).

HPV gene expression in high-grade premalignant disease or cancer appears restricted to the early genes, possibly due to cellular differentiation arrest induced by the viral E6 and E7 genes. In comparison to active HPV infection, E6 and E7 gene control in cancer is deranged by mutations in the viral URR and, in integrated viral fragments, by the disruption of the viral E2 gene, stabilization of E6 and E7 mRNAs, and influences at the cellular integration site.

Because the E2 gene is disrupted or inactivated in integrated HPV fragments in invasive cervical carcinomas (Cullen et al., 1991; Durst et al., 1985; Matsukura et al., 1989; Schneider-Gadicke et al., 1986; Schwarz et al., 1985; Wilczynski et al., 1988), it has been predicted that loss of E2 bestows a selective growth advantage to the infected cell because of uncontrolled E6 and E7 expression (Schneider-Gadicke et al., 1986; Schwarz et al., 1985). Indeed, cervical cells containing replicating HPV genomes rapidly segregate and are outgrown in culture by cells that contain integrated viral genomes (Jeon et al., 1995), but the underlying mechanism(s) have remained unclear until recently. The full-length HPV 16 E2 gene products are strong transcriptional activators comparable to HPV 1 E2 at some viral as well as at simple, synthetic promoters (Demeret et al., 1994; Ushikai et al., 1994).

Genes E6 and E7 are considered to have oncogenic activity. The encoded proteins interact with and disturb the physiologic functions of cellular proteins that are involved in cell cycle control. The E6/E7 proteins of HPV 16, 18 or related types are most efficient in this regard. Some of these activities lead to genetic instability of the persistently infected human cell. This enhances the probability of mutations in cellular proto-oncogenes and tumor suppressor genes and thus contributes to tumor progression. Mutations in cellular genes devoted to the intracellular surveillance of HPV infections, integration of viral DNA, and deletions or mutations of viral transcription control sequences lead to a significantly increased expression of the E6/E7 genes, which is a consistent characteristic of high-grade intraepithelial neoplasia and cancers. The genetic instability caused by viral oncoproteins and the autocatalytic increase in oncoprotein expression caused by mutations in the viral and cellular genome identify the virus as a major driving force of progression to carcinoma.

B. p53

The p53 gene encodes a 375-amino-acid phosphoprotein that can form complexes with viral proteins such as large-T antigen and E1B. The protein is found in normal tissues and cells, but at concentrations which are minute by comparison with many transformed cells or tumor tissue. Interestingly, wild-type p53 appears to be important in regulating cell growth and division. Overexpression of wild-type p53 has been shown in some cases to be anti-proliferative in human tumor cell lines. Thus p53 can act as a negative regulator of cell growth (Weinberg, 1991) and may directly suppress uncontrolled cell growth or indirectly activate genes that suppress this growth. Thus, absence or inactivation of wild-type p53 may contribute to transformation. However, some studies indicate that the presence of mutant p53 may be necessary for full expression of the transforming potential of the gene.

Although wild-type p53 is recognized as a centrally important growth regulator in many cell types, its genetic and biochemical traits appear to have a role as well. Mis-sense mutations are common for the p53 gene and are essential for the transforming ability of the oncogene. A single genetic change prompted by a point mutation can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991). Sisk et al. (2002) has shown that p53 mutation and HPV infection are potential risk factors for HNSCC. The presence of HPV was found to confer a survival advantage among HNSCC patients, particularly when p53 was wild-type.

Casey and colleagues have reported that transfection of DNA encoding wild-type p53 into two human breast cancer cell lines restores growth suppression control in such cells (Casey et al., 1991). A similar effect has also been demonstrated on transfection of wild-type, but not mutant, p53 into human lung cancer cell lines (Takahasi et al., 1992). The wild-type p53 appears dominant over the mutant gene and will select against proliferation when transfected into cells with the mutant gene. Expression of the transfected p53 does not affect the growth of normal cells with endogenous p53. Thus, such constructs might be taken up by normal cells without adverse effects. Introduction of wild-type p53 into a cervical cancer cell line in vitro resulted in growth suppression and induction of apoptosis (Hamada et al, 1996).

It now has been observed that p53 gene therapy of cancers may be effective regardless of the p53 status of the tumor cell. Surprisingly, therapeutic effects have been observed when a viral vector carrying the wild-type p53 gene is used to treat a tumor, the cells of which express a functional p53 molecule. This result would not have been predicted based on the current understanding of how tumor suppressors function. It also is surprising given that normal cells, which also express a functional p53 molecule, are apparently unaffected by expression of high levels of p53 from a viral construct. This raises the possibilitity that p53 gene therapy may be more broadly applicable to the treatment of cancers than was initially suspected.

Throughout this application, the term “p53” is intended to refer to the exemplified p53 molecules as well as all p53 homologues from other species. “Wild-type” and “mutant” p53 refer, respectively, to a p53 gene expressing normal tumor suppressor activity and to a p53 gene lacking or having reduced suppressor activity and/or having transforming activity. Thus “mutant” p53 are not merely sequence variants but rather, are those variants showing altered functional profiles.

While tumors containing a mutated p53 gene are a preferred target according to the present invention, the utility of the claimed p53 expression vectors extends to the treatment of tumors having wild-type or functional p53. Though the mechanism is not completely understood, the inventor has determined that expression of exogenous p53 through gene transfer can suppress HPV immortalization and carcinogen transformation in oral keratinocytes and HNSCC in vitro. This phenomenon is not limited to HNSCC and HPV-immortalized and carcinogen-transformed oral keratinocytes, but may be applied to a wide variety of malignancies including gliomas, sarcomas, carcinomas, leukemias, lymphomas and melanoma, including tumors of the skin, liver, testes, bone, brain, pancreas, stomach, liver, lung, ovary, cervix, vagina, uterus, breast, colon, prostate and bladder.

1. p53 Polypeptides

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity, i.e., tumor suppression or tumor growth inhibition or induction of apoptosis. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

Amino acid sequence variants of p53 also are encompassed by the present invention. Amino acid sequence variants of the polypeptide can be substitutional variants or insertional variants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide.

Amino acid substitutions are generally based on the relative similarity of the amino acid side-chain substituents, or example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated by reference herein). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is understood that an amino acid can be substituted for another having a similar hydrophilicity value and still obtain a biologically equivalent protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

2. p-53 Encoding Polynucleotides

The polynucleotides according to the present invention may encode an entire p53 gene, a functional p53 protein domain, or any p53 polypeptide. The polynucleotides may be derived from genomic DNA, i.e., cloned directly from the genome of a particular organism. In other embodiments, however, the polynucleotides may be complementary DNA (cDNA). cDNA is DNA prepared using messenger RNA (mRNA) as a template. Thus, a cDNA does not contain any interrupted coding sequences and usually contains almost exclusively the coding region(s) for the corresponding protein. In other embodiments, the polynucleotide may be produced synthetically.

It may be advantageous to combine portions of the genomic DNA with cDNA or synthetic sequences to generate specific constructs. For example, where an intron is desired in the ultimate construct, a genomic clone will need to be used. Introns may be derived from other genes in addition to p53. The cDNA or a synthesized polynucleotide may provide more convenient restriction sites for the remaining portion of the construct and, therefore, would be used for the rest of the sequence.

It is contemplated that natural variants of p53 exist that have different sequences than those disclosed herein. Thus, the present invention is not limited to use of the provided polynucleotide sequence for p53 but, rather, includes use of any naturally-occurring variants. The present invention also encompasses chemically synthesized mutants of these sequences.

Another kind of sequence variant results from codon variation. Because there are several codons for most of the 20 normal amino acids, many different DNA's can encode the p53. Reference to the following table will allow such variants to be identified.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have between about 50% and about 75%, or between about 76% and about 99% of nucleotides that are identical to the nucleotides disclosed herein will be preferred. Sequences that are within the scope of “a p53-encoding polynucleotide” are those that are capable of base-pairing with a polynucleotide segment set forth above under intracellular conditions.

As stated above, although the p53 encoding sequences may be full length genomic or cDNA copies, or large fragments thereof. The present invention also may employ shorter oligonucleotides of p53. Sequences of 17 bases long should occur only once in the human genome and, therefore, suffice to specify a unique target sequence. Although shorter oligomers are easier to make and increase in vivo accessibility, numerous other factors are involved in determining the specificity of base-pairing. Both binding affinity and sequence specificity of an oligonucleotide to its complementary target increases with increasing length. It is contemplated that oligonucleotides of 8, 9, 10, 11, 12, 13, 14, 15, 16, 17, 18, 19 or 20 base pairs will be used, for example, in the preparation of p53 mutants and in PCR reactions.

Any sequence of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length.

In certain embodiments, one may wish to employ constructs which include other elements, for example, those which include C-5 propyne pyrimidines. Oligonucleotides which contain C-5 propyne analogues of uridine and cytidine have been shown to bind RNA with high affinity (Wagner et al., 1993).

C. EXPRESSION CASSETTES

1. Overview

Throughout this application, the term “expression cassette” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a p53 gene and translation of a p53 mRNA into a p53 protein product.

2. Promoters and Enhancers

In order for the expression cassette to effect expression of at least a p53 transcript, the polynucleotide encoding the p53 polynucleotide will be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

The promoter will be one which is active in the target cell. For instance, where the cell in the specific embodiment is a keratinocyte, the promoter will be one which has activity in a keratinocyte. Similarly, where the cell is an epithelial cell, skin cell, mucosal cell or any other cell that can undergo transformation by a papillomavirus, the promoter used in the embodiment will be one which has activity in that particular cell type.

A promoter may be one naturally associated with a gene or sequence, as may be obtained by isolating the 5′-non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCRTM, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The particular promoter that is employed to control the expression of a p53 polynucleotide is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used to obtain high level expression of the p53 polynucleotide. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosine (melanoma), alpha-fetoprotein and albumin (liver tumors), CC10 (lung tumors) and prostate-specific antigen (prostate tumor) will permit tissue-specific expression of p53 polynucleotides. Table 2 lists several promoters/elements which may be employed, in the context of the present invention, to regulate the expression ofp53 constructs. This list is not intended to be exhaustive of all the possible elements involved in the promotion of p53 expression but, merely, to be exemplary thereof.

TABLE 2

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe et |
| | al., 1988; Rosen et al., 1988; Berkhout et al., 1989; |
| | Laspia et al., 1989; Sharp et al., 1989; Braddock et |
| | al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and continguous, often seeming to have very similar modular organization.

Additionally, any promoter/enhancer combination (as per the Eukaryotic Promoter Data Base EPDB) could also be used to drive expression of a p53 construct. Use of a T3, T7, or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional expression vector.

Further selection of a promoter that is regulated in response to specific physiologic signals can permit inducible expression of the p53 construct. For example, with the polynucleotide under the control of the human PAI-1 promoter, expression is inducible by tumor necrosis factor. Table 3 provides examples of inducible elements, which are regions of a nucleic acid sequence that can be activated in response to a specific stimulus.

TABLE 3

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et al., 1983; |
| | | Ponta et al., 1985; Sakai et |
| | | al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid | Thyroid Hormone | Chatterjee et al., 1989 |
| Stimulating | | |
| Hormone α Gene | | |

3. Markers

In certain embodiments of the invention, the delivery of an expression cassette in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenical acetyltransferase (CAT)(prokaryotic) may be employed. Immunologic markers can also be employed. The selectable marker employed is not believed to be important, so long as it is capable of being expressed along with the polynucleotide encoding p53. Further examples of selectable markers are well known to one of skill in the art.

4. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

5. IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819).

6. Multiple Cloning Sites

Expression cassettes can include a multiple cloning site (MCS), which is a nucleic acid region that contains multiple restriction enzyme sites, any of which can be used in conjunction with standard recombinant technology to digest the vector. See Carbonelli et al. (1999); Levenson et al. (1998); Cocea (1997). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997).

7. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

8. Other Expression Cassette Components

In preferred embodiments of the present invention, the expression cassette comprises a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, expression vectors need not be viral but, instead, may be any plasmid, cosmid or phage construct that is capable of supporting expression of encoded genes in mammalian cells, such as pUC or Bluescript™ plasmid series.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

In certain embodiments of the invention, a treated cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

D. GENE TRANSFER

1. Viral Vectors

A “viral vector” is meant to include those constructs containing viral sequences sufficient to (a) support packaging of the p53 expression cassette and (b) to ultimately express a recombinant gene construct that has been cloned therein.

a. Adenoviral Vectors

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors.

Adenoviruses are currently the most commonly used vector for gene transfer in clinical settings. Among the advantages of these viruses is that they are efficient at gene delivery to both nondividing an dividing cells and can be produced in large quantities. In many of the clinical trials for cancer, local intratumor injections have been used to introduce the vectors into sites of disease because current vectors do not have a mechanism for preferential delivery to tumor. In vivo experiments have demonstrated that administration of adenovirus vectors systemically resulted in expression in the oral mucosa (Clayman et al., 1995). Topical application of Ad-βgal and Ad-p53-FLAG on organotypic raft cultures has demonstrated effective gene transduction and deep cell layer penetration through multiple cell layers (Eicher et al., 1996). Therefore, gene transfer strategy using the adenoviral vector is potentially feasible in patients at risk for lesions and malignancies involving genetic alterations inp53.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Retroviral Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

c. AAV Vectors

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Herpesvirus Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975). The expression of a genes, the first set of genes to be expressed after infection, is enhanced by the virion protein number 16, or α-transinducing factor (Post et al., 1981; Batterson and Roizman, 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

e. Vaccinia Virus Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

f. Other Viral Vectors

Other viral vectors may be employed as constructs in the present invention. Vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependeht packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

g. Gene Delivery Using Modified Viruses

A p53-encoding nucleic acid may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Nonviral Vectors

a. Examples of Non-Viral Vectors

Several non-viral methods for the transfer of expression vectors into cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 19779) and liofectamine-DNA complexe, cell sonication (Fechheimer et al., 1987), gene bombardment using high velocity microprojectiles (Yang et al., 1990), polycations (Bousssif et al., 1995) and receptor-mediated transfection (Wu and Wu, 1987; Wu and Wu, 1988). Some of these techniques may be successfully adapted for in vivo or ex vivo use.

In one embodiment of the invention, the adenoviral expression cassette may simply consist of naked recombinant vector. Transfer of the construct may be performed by any of the methods mentioned above which physiclaly or chemically permeabilize the cell membrane. For example, Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a p53 construct may also be transferred in a similar manner in vivo.

Another embodiment of the invention for transferring a naked DNA expression vector into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990). This may require surgical exposure of the tissue or cells, to eliminate any intervening tissue between the gun and the target organ. DNA encoding a p53 construct may be delivered via this method.

In other embodiments of the present invention, the transgenic construct is introduced to the cells using calcium phosphate co-precipitation. Mouse primordial germ cells have been transfected with the SV40 large T antigen, with excellent results (Watanabe et al., 1997). Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

Further embodiments of the present invention include the introduction of the nucleic acid construct by direct microinjection or sonication loading. Direct microinjection has been used to introduce nucleic acid constructs into Xenopus oocytes (Harland and Weintraub, 1985), and LTK⁻ fibroblasts have been transfected with the thymidine kinase gene by sonication loading (Fechheimer et al., 1987).

b. Lipid and Liposome Non-Viral Vectors

In a further embodiment of the invention, the gene construct may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (WO 98/07408). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these liposomes include a positive p, colloidal stabilization by cholesterol, two dimensional DNA packing and increased serum stability.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

D. CANCER OF THE HEAD AND NECK

The term “cancer” as used herein is defined as a tissue of uncontrolled growth or proliferation of cells, such as a tumor. Head and neck cancer is the term given to a variety of malignant tumors that may occur in the head and neck region: the oral cavity (including the tissues of the lip or mouth such as the tongue, the gums, the lining of the cheeks and lips, the bottom of the mouth, the hard and soft palate and the retromolar trigone); the pharynx (including the hypopharynx, nasopharynx and oropharynx, also called the throat); paranasal sinuses (including the frontal sinuses above the nose, the maxillary sinuses in the upper part of either side of the upper jawbone, the ethmoid sinuses just behind either side of the upper nose, and the sphenoid sinus behind the ethmoid sinus in the center of the skull) and nasal cavity; the larynx (also called the voicebox); thyroid gland (including cancers of the thyroid which are papillary, follicular, medullary and anaplastic); parathyroid gland; salivary glands (including the major clusters of salivary glands found below the tongue, on the sides of the face just in front of the ears, and under the jawbone); lesions of the skin of the face and neck and the cervical lymph nodes; and metastatic squamous neck cancer with occult primary.

Although the percentage of oral and head and neck cancer patients in the United States is only about 5% of all cancers diagnosed, the importance of this disease is heightened by the fact that functional and aesthetic problems are commonly associated with this type of cancer and its treatment. Estimates indicate that there are more than 500,000 survivors of oral and head and neck cancer living in the United States today. Coping with this type of cancer can be extremely difficult. Not only can the disease be life-threatening, but many patients must also endure alterations in facial and neck appearance, as well as alterations in speech, sight, smell, chewing, swallowing and taste perception.

Normal aerodigestive tract mucosa is transformed into damaged epithelial cells, squamous hyperplasia, and then premalignant cells or squamous intraepithelial neoplasia (SIN). Squamous intraepithelial neoplasia includes squamous hyperplasia, mild, moderate and severe dysplasia. Afterward, SIN will evolve into early cancer. Cancer cells subsequently will progress to become more aggressive and subsequently metastasize (advanced cancer). A genetic progression model has been proposed in HNSCC (Califano et al., 1996). The earliest genetic alteration is loss of chromosome 9p (Mao et al. 1996) and 16p (Papadimitrakopoulou et al., 1997), followed by loss of 3p and 17p (Mao et al. 1996), mutations in p53 (Boyle et al., 1993) and DNA ploidy aberrations (Munck-Wikland et al., 1997). Tobacco carcinogens induce these genetic alterations in HNSCC.

Head and neck cancers can arise from squamous cell carcinomas (SCC), which are the second most common form of skin cancer. They occur in men more often than women and originate primarily in skin exposed to the sun in a dose-dependent manner. SCCs are likely derived from keratinocytes located near the skin surface. Aneuploidy is common in this type of cancer, as is the presence of p53 mutations. SCC may occur anywhere on the skin, although it may arise on the mucosal membranes of the mouth, nose, lips, throat, eyelids, lining of the breathing tubes, anus, cervix, etc.

E. THERAPIES

1. Overview

The present invention contemplates methods to inhibit the growth of a papillomavirus-transformed cell in a hyperplastic lesion in a subject by topical delivery of a growth-inhibiting amount of an expression cassette encoding a p53 polypeptide in a pharmaceutical preparation suitable for topical delivery. In preferred embodiments, inhibition of growth can include slowing or halting of growth, reduction of the size of the lesion, induction of apoptosis of the lesion, or induction of an immune response against the cells of the lesion. The present invention also contemplates compositions to be used for the inhibition of growth of a papillomavirus-transformed cell in a hyperplastic lesion in a subject of an expression cassette encoding a promoter and p53 polypeptide in an appropriate pharmaceutical carrier. The compositions include a mouthwash, douche solution formulated for vaginal delivery, suppository for anal or vaginal delivery, cream formulated for topical, anal, or vaginal delivery, solution formulated for hypospray, or an aerosolized suspension. In addition, the present invention contemplates methods for suppressing or preventing papillomavirus-mediated transformation of a keratinocyte in a subject by administering a composition comprising an expression cassette encoding a promoter and p53 polypeptide in a pharmaceutical preparation suitable for topical delivery.

2. Examples of Hyperplastic Lesions

Examples of hyperplastic lesions that are contemplated for treatment include, but are not limited to, squamous cell hyperplastic lesions, premalignant epithelial lesions, psoriatic lesions, cutaneous warts, periungual warts, anogenital warts, epidermdysplasia verruciformis, intraepithelial neoplastic lesions, focal epithelial hyperplasia, conjunctival papilloma, conjunctival carcinoma, or squamous carcinoma lesion. Treatment of. carcinomas related to papillomavirus is also contemplated, including but not limited to cancers of the head and neck, cervix, anus, penis. The lesion include, but is not limited to, cells such as keratinocytes, epithelial cells, skin cells, and mucosal cells. The subject to be treated includes, but is not limited to, humans and mammals.

3. Growth Inhibition Defined

“Inhibiting the growth” of a hyperplastic lesion is broadly defined and includes, for example, a slowing or halting of the growth of the lesion. Inhibiting the growth of a lesion can also include a reduction in the size of a lesion or induction of apoptosis of the cells of the lesion. The term “induction of apoptosis” as used herein refers to a situation wherein a drug, toxin, compound, composition or biological entity bestows apoptosis, or programmed cell death, onto a cell. In a specific embodiment, the cell is a tumor cell. In another embodiment the tumor cell is a head and neck cancer cell, a squamous cell carcinoma, a cervical cancer cell, or a cell of an anogenital wart. In further embodiments, the cell is a keratinocyte, an epithelial cell, a skin cell, a mucosal cell, or any other cell that can undergo transformation by a papillomavirus. Growth of a lesion can be inhibited by induction of an immune response against the cells of the lesion.

4. Compositions for Topical Administration

a. Topical Administration Defined

In the context of the claimed invention, “topical administration” is defined to include administration to the exterior surface of the body such as the skin, eye or anus, administration to the surface of an internal area of the body such as the oral mucosa, cervix or vagina, or administration to the surface of the bed of an excised lesion in any of these areas (i.e., the surgical bed of an excised pharyngeal HNSCC or an excised cervical carcinoma).

b. Compositions Using Viral Vectors

Where clinical application of an viral expression vector according to the present invention is contemplated, it will be necessary to prepare the complex as a pharmaceutical composition appropriate for the intended application. Generally, this will entail preparing a pharmaceutical composition that is essentially free of pyrogens, as well as any other impurities that could be harmful to humans or animals. One also will generally desire to employ appropriate salts and buffers to render the complex stable and allow for complex uptake by target cells.

c. Pharmaceutical Compositions

The phrase “pharmaceutical preparation suitable” and “formulated” refer to molecular entities and compositions that do not produce an adverse, allergic or other untoward reaction when administered to an animal, or a human, as appropriate. As used herein, “pharmaceutical preparation” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the composition. In addition, the composition can include supplementary inactive ingredients. For instance, the composition for use as a mouthwash may include a flavorant or the composition may contain supplementary ingredients to make the formulation timed-release.

Aqueous compositions of the present invention comprise an effective amount of the expression cassette, dissolved or dispersed in a pharmaceutically acceptable carrier or acqueous medium. Such compositions also are referred to as inocula. Examples of aqueous compositions include a mouthwash or mouthrinse, douche solution for vaginal use, spray or aerosol, or ophthalmic solution.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The expression cassettes and delivery vehicles of the present invention may include classic pharmaceutical preparations. Administration of therapeutic compositions according to the present invention will be via any common route so long as the target tissue is available via that route. For example, this includes oral, nasal, buccal, anal, rectal, vaginal, or topical ophthalmic. Such compositions would normally be administered as pharmaceutically acceptable compositions that include physiologically acceptable carriers, buffers or other excipients.

The therapeutic and preventive compositions of the present invention are advantageously administered in the form of liquid solutions or suspensions; solid forms suitable for solution in, or suspension in, liquid prior to topical use may also be prepared. A typical composition for such purpose comprises a pharmaceutically acceptable carrier. For instance, the composition may contain 10 mg, 25 mg, 50 mg or up to about 100 mg of human serum albumin per ml of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Preservatives include antimicrobial agents, anti-oxidants, chelating agents and inert gases. The pH and exact concentration of the various components of the pharmaceutical composition are adjusted according to well-known parameters.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions such as mouthwashes and mouthrinses, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

For oral administration the expression cassette of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient also may be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

One may also use solutions and/or sprays, hyposprays, aerosols and/or inhalants in the present invention for administration. One example is a spray for administration to the aerodigestive tract. The sprays are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Formulations for other types of administration that is topical include, for example, a cream, suppository, ointment or salve.

d. Dosage

An effective amount of the therapeutic or preventive agent is determined based on the intended goal, for example (i) inhibition of growth of a hyperplastic lesion or (ii) induction of an immune response against a hyperplastic lesion.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

The quantity to be administered, both according to number of treatments and dose, depends on the subject to be treated, the state of the subject and the protection desired. Precise amounts of the therapeutic composition also depend on the judgment of the practitioner and are peculiar to each individual.

In certain embodiments, it may be desirable to provide a continuous supply of the therapeutic compositions to the patient. For topical administrations, repeated application would be employed. For various approaches, delayed release formulations could be used that provide limited but constant amounts of the therapeutic agent over an extended period of time. For internal application, continuous perfusion of the region of interest may be preferred. This could be accomplished by catheterization, post-operatively in some cases, followed by continuous administration of the therapeutic agent. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

5. Treatment of Artificial and Natural Body Cavities

One of the prime sources of recurrent HNSCC is the residual, microscopic disease that remains at the primary tumor site, as well as locally and regionally, following tumor excision. In addition, there are analogous situations where natural body cavities are seeded by microscopic tumor cells. The effective treatment of such microscopic disease would present a significant advance in therapeutic regimens.

Thus, in certain embodiments, a cancer may be removed by surgical excision, creating a “cavity.” Both at the time of surgery and thereafter (periodically or continuously), the therapeutic composition of the present invention is administered to the body cavity. This is, in essence, a “topical” treatment of the surface of the cavity. The volume of the composition should be sufficient to ensure that the entire surface of the cavity is contacted by the expression cassette.

In one embodiment, administration simply will entail injection of the therapeutic composition into the cavity formed by the tumor excision. In another embodiment, mechanical application via a sponge, swab or other device may be desired. Either of these approaches can be used subsequent to the tumor removal as well as during the initial surgery. In still another embodiment, a catheter is inserted into the cavity prior to closure of the surgical entry site. The cavity may then be continuously perfused for a desired period of time.

In another form of this treatment, the “topical” application of the therapeutic composition is targeted at a natural body cavity such as the mouth, pharynx, esophagus, larynx, trachea, pleural cavity, peritoneal cavity, or hollow organ cavities including the bladder, colon or other visceral organ. In this situation, there may or may not be a significant, primary tumor in the cavity. The treatment targets microscopic disease in the cavity, but incidentally may also affect a primary tumor mass if it has not been previously removed or a pre-neoplastic lesion which may be present within this cavity. Again, a variety of methods may be employed to affect the “topical” application into these visceral organs or cavity surfaces. For example, the oral cavity in the pharynx may be affected by simply oral swishing and gargling with mouthwashes or mouth rinses. However, topical treatment within the larynx and trachea may require endoscopic visualization and topical delivery of the therapeutic composition, or administration via a spray or aerosol formulation. Visceral organs such as the bladder or colonic mucosa may require indwelling catheters with infusion or again direct visualization with a cystoscope or other endoscopic instrument. Body cavities may also be accessed by indwelling catheters or surgical approaches which provide access to those areas.

6. Tracers to MonitorpS3 Expression Following Administration

Because destruction of microscopic tumor cells cannot be observed, it is important to determine whether the target site has been effectively contacted with the expression construct. This may be accomplished by identifying cells in which the expression construct is actively producing the p53 product. It is important, however, to be able to distinguish between the exogenous p53 and that present in tumor and nontumor cells in the treatment area. Tagging of the exogenous p53 with a tracer element would provide definitive evidence for expression of that molecule and not an endogenous version thereof. Thus, the methods and compositions of the claimed invention may involve tagging of the p53 encoded by the expression cassette with a tracer element.

One such tracer is provided by the FLAG biosystem (Hopp et al., 1988). The FLAG polypeptide is an octapeptide (AspTyrLysAspAspAspAspLys) and its small size does not disrupt the expression of the delivered gene therapy protein. The coexpression of FLAG and the protein of interest is traced through the use of antibodies raised against FLAG protein.

Other immunologic marker systems, such as the 6×His system (Qiagen) also may be employed. For that matter, any linear epitope could be used to generate a fusion protein with p53 so long as (i) the immunologic integrity of the epitope is not compromised by the fusion and (ii) the functional integrity of p53 is not compromised by the fusion.

7. Preventive Therapies

The best strategy for patients with HNSCC is prevention by either smoking cessation or therapeutic intervention, such as chemoprevention. After patients with HNSCC are cured, they have a significant (30-40%) chance of having a second primary tumor (Khuri et al., 1997). Chemoprevention of high-risk populations may reduce the development of a second primary tumor and improve survival (Khuri et al., 1997). The mucosa of the upper aerodigestive tract (UADT) is at risk for developing second primary tumors by micrometastasis (Bedi et al., 1996) or by field cancerization (Lydiatt et al., 1998). Because genetic alterations are found in histologically and clinically normal appearing mucosal tissue, these cells can progress to form a second primary tumor. These precancerous cells therefore are targets for therapeutic gene transfer. Arresting the G1-phase of the cell cycle in preneoplastic cells may halt cellular progression. If overexpression of p53 can suppress preneoplastic UADT cells, then p53 gene transfer may prevent the development of HNSCC.

This same strategy can be applied to other hyperplastic lesions that are causally related to HPV. Populations at risk can include those subjects with a history of a previous hyperplastic lesion presumed to be causally related to HPV or those who have some other risk factor for development of the hyperplastic lesion.

The quantity of pharmaceutical composition to be administered, according to dose, number of treatments and duration of treatments, depends on the subject to be treated, the state of the subject, the nature of the previous hyperplastic lesion and the protection desired. Precise amounts of the therapeutic composition also depend on the judgment of the practitioner and are peculiar to each individual. For example, the frequency of application of the composition can be once a day, twice a day, once a week, twice a week, or once a month. Duration of treatment may range from one month to one year or longer. Again, the precise preventive regimen will be highly dependent on the subject, the nature of the risk factor, and the judgment of the practitioner.

F. SECONDARY ANTI-HYPERPLASTIC THERAPIES

1. General

In an embodiment of the present invention there is a method of inhibiting the growth of a papillomavirus-transformed cell in a hyperplastic lesion utilizing a growth inhibiting amount of a composition comprising an expression cassette encoding a p53 polypeptide. In one embodiment of the claimed invention, the hyperplastic lesion is a cancer, such as a squamous cell carcinoma. In another embodiment of the claimed invention, the treatment of the hyperplastic lesion occurs in conjunction with secondary antihyperplastic therapy. Examples of secondary hyperplastic therapy include chemotherapy, radiotherapy, immunotherapy, phototherapy, cryotherapy, toxin therapy, hormonal therapy or surgery. Thus, the claimed invention contemplates use of the claimed methods and compositions in conjunction with standard anti-cancer therapies. The patient to be treated may be an infant, child, adolescent or adult.

A wide variety of cancer therapies, known to one of skill in the art, may be used in combination with the compositions of the claimed invention. Some of the existing cancer therapies and chemotherapeutic agents are described below. One of skill in the art will recognize the presence and development of other anticancer therapies which can be used in conjugation with the compositions comprising p53 expression cassettes and will further recognize that the use of the secondary antihyperplastic therapy of the claimed invention will not be restricted to the agents described below.

In order to increase the effectiveness of a an expression construct encoding a p53 polypeptide, it may be desirable to combine these compositions with other agents effective in the treatment of hyperproliferative disease. These compositions would be provided in a combined amount effective to kill or inhibit proliferation of the cell. This process may involve contacting the cells with the expression construct and the agent(s) or second factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, p53 therapy is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

2. Radiotherapy

Radiotherapy include radiation and waves that induce DNA damage for example, y-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In the context of the present invention radiotherapy may be used in addition to using the tumor cell specific-peptide of the invention to achieve cell-specific cancer therapy.

3. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, in the context of the present invention surgery may be used in addition to using the tumor cell specific-peptide of the invention to achieve cell-specific cancer therapy.

In the case of surgical intervention, the compositions of the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising a p53-encoding construct. The perfusion may be continued post-resection, for example, by leaving a catheter implanted at the site of the surgery. Periodic post-surgical treatment also is envisioned.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently may be described in terms of plaque forming units (pfi) for a viral construct. Unit doses range from 10³, 10⁴, 10⁵, 10⁶, 10⁷, 10⁸, 10⁹, 10¹⁰, 10¹¹, 10¹², 10¹³ pfu and higher.

4. Chemotherapeutic Agents

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate or any analog or derivative variant thereof. The term “chemotherapy” as used herein is defined as use of a drug, toxin, compound, composition or biological entity which is used as treatment for cancer. These can be, for example, agents that directly cross-link DNA, agents that intercalate into DNA, and agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms, these compounds are administered through bolus injections intravenously at doses ranging from 25-75 mg/m² at 21 day intervals for adriamycin, to 35-100 mg/m² for etoposide intravenously or orally.

5. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with p53 therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

6. Genes

In yet another embodiment, the secondary treatment is a gene therapy in which a non-p53 expression cassette is administered before, after, or at the same time as a p53 expression cassette. Delivery may comprise use of a vector encoding p53 in conjunction with a second vector encoding an additional gene product. Alternatively, a single vector encoding both genes may be used. A variety of secondary gene therapy proteins are envisioned within the invention, some of which are described below.

7. Other Cancer Therapies

Examples of other cancer therapies include phototherapy, cryotherapy, toxin therapy, or hormonal therapy. One of skill in the art would know that this list is not exhaustive of the types of treatment modalities available for cancer and other hyperplastic lesions.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Lines. Immortalized human gingival keratinocytes (IHGK) cells are oral keratinocytes that have been immortalized with HPV16 (Oda et al., 1996); these cells proliferate only in enriched keratinocyte growth media (DK-SFM; Gibco-BRL, Grand Island, N.Y.) containing low amounts of calcium and no serum. These cells have features of preneoplasia (Oda et al. 1996; Yoo et al., 2000).

IHGK cells were examined at passages less than 100 because spontaneous p53 mutations are observed at passages later than 130 (Oda et al., 1996). IHGK cells were transformed with a carcinogen, 4-(methyllnitrosamino)-1-(30pyridyl)-1-butanone (NNK), by a 5-week exposure to a media containing 36 μg/ml of NNK. Then, the transformed cells were selected with Dulbecco minimum essential medium (DMEM) supplemented with 10% fetal calf serum (FCS) media because HNSCC cell lines, but not IHGK cells, grow in serum containing media. The selected cell line was designated IHGKN. Two HNSCC cell lines, HN12 and HN30, were grown in DMEM with 10% FCS. The p53 gene is mutated in HN12 whereas HN30 has a wild-typep53 gene (Yeudall et al., 1997). HN30 and HN12 did not express p16 or p14 because of either a mutation or a homozygous deletion (Yeudall et al., 1994; Yoo et al., 2000).

Adenoviral Constructs and Transduction of Cells. Ad-p53 (Ad5CMV-p53; RPR/INGN 201) and Ad-βGal were obtained from Introgen Therapeutics, Inc. and stored at −80° C. Before use, the viruses were thawed slowly on ice. The virus constructs were diluted in culture media to desired concentrations. Serial dilutions were prepared to make viral particle to cell (VPC) ratios of 100, 500, 1000, 5000, and 10,000. Cells were plated to reach 70-80% confluence for all experiments. Both Ad-p53 or Ad-βGal transductions were performed by adding new culture media to either 6-well plates or 100 mm2 plates and then adding adenoviruses. β-Galactosidase activity was measured by X-gal staining. Cells were fixed (phosphate-buffered saline (PBS)+0.5% (v/v) glutaraldehyde) for 10 minutes and then washed twice with PBS. Cells were stained with X-Gal solution (2 mM MgCl₂, 1 mg/mL X-Gal, and 5 mM potassium ferrocyanide in PBS) for 24 hours.

Proliferation Assay. Proliferation rates were determined by measuring the uptake of ³H-thymidine in triplicate. In each well of a 96-well plate, 15,000 cells (70-80% confluence) were plated with varying concentrations of Ad-βGal or Ad-p53 for a total volume per well of 200 ItL. After 24, 48, and 72 hours, ³H-thymidine was added to each well to yield a final concentration of 1% (v/v). After 24 hours of incubation, cells were harvested onto a filter. After the filter was dried for 4 hours, a scintillation cocktail was added. A Trilux beta counter (Wallac, Gaithersburg, Md.) was used to determine the amount of ³H-thymidine incorporated into the dividing cells. The inhibition of proliferation was calculated using a ratio: (cpm^{Ad-p53})/cmp^{Ad-βGal.}

Cell Cycle Distribution. Cells (4×10⁵) were plated in 6-well plates with 1 mL DK-SFM and allowed to reach 70-80% confluence before transduction. The virus and cells then were incubated overnight, and then 48 hours later cells were ethanol-fixed and further incubated with propidium iodide (20 μg/mL) and ribonuclease (200 μg/mL) for 20 minutes at 37° C. Cell cycle distribution was measured using flow cytometry (FACScan; Becton Dickinson, Bedford, Mass.). At least 10,000 events per sample were analyzed. ModFit LT (Verity Software House, Topsham, Me.) cytologic software program was used for data analysis. ModFit LT uses mathematic models to fit data from FACS to generate curves of each cell cycle phase and area under the curve. The percentage of cells in G0/G1, S, and G2/M phases of these cells then were determined.

Western Blot. Cells (1.2×10⁶) were plated (100 mm²) in 6 mL DK-SFM. After 48 hours of incubation with the viruses, the cells were washed with PBS. Total cell lysates were prepared by sonicating and incubating the cells in RIPA buffer (150 mM NaCl, 1% Triton X-100, 1% sodium deoxycholate, 0.4% sodium dodecyl sulfate (SDS), 20 mM ethylenediamine tetraacetic acid, and 50 mM Tris, pH 7.4) for 1 hour at 4° C. Equal amounts of protein from each sample were subjected to 7-14% SDS polyacrylamide gel electrophoresis and transferred to a nitrocellulose membrane (Bio-Rad, Hercules, Calif.). The membrane was blocked with Blotto-Tween (10% nonfat milk, 0.05% Tween 20, 0.9% NaCl, and 50 mM Tris, pH 7.5) and incubated with primary antibodies against p21 (PharMingen, San Diego, Calif.) or p53 (Pab 240; PharMingen). A secondary antibody, horseradish peroxidase-conjugated immunoglobulin G was incubated with membranes and developed according to Amersham's enhanced chemiluminescence protocol (ECL; Amersham, Piscataway, N.J.).

Apoptosis. For analysis of apoptosis, annexin V binding and dead cells (propidium iodide staining) were measured after Ad-p53 or Ad-βgal was applied. Flow cytometry (FACS; Becton Dickinson) was used to measure the binding of Annexin V fluorescein isothiocyanate (FITC; Chemicon International Inc.) to phosphatidyl serine, which is translocated to the outer membrane of the cell during the early states of apoptosis. Cells dying because of nonapoptotic pathways were excluded by concurrent incubation with propidium iodide. The data collected by FACS were plotted by propidium iodide versus Annexin V FITC dot plot using WinMDI 2.7 software (Becton Dickinson).

### Example 2

### Results

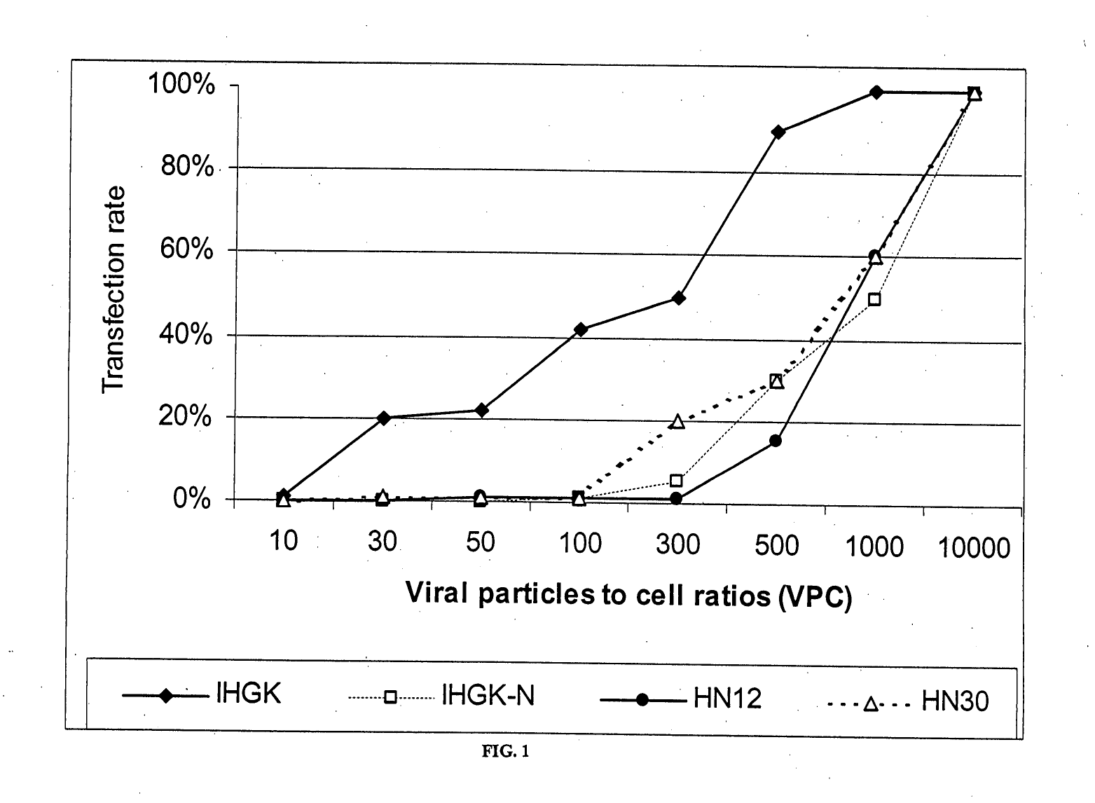

Effective Ratio of Viral Particles per Cell. After transduction of IHGK, IHGKN, HN12, and HN30 with Ad-βgal at 100, 500, 1000, 5000, and 10,000 VPC, βgal activity was measured (FIG. 1). IHGK cells were more efficiently transduced at lower VPC ratios than IHGKN, HN12, and HN30, which had similar transduction efficiencies. At VPC ratios of 1000, IHGK cells reached 100% transduction efficiency whereas all other cells required a VPC ratio of 10,000. The increased transduction rate in IHGK cells may be due to expression of coxsackie-adenovirus receptor (CAR) and integrin as previously reported (Li et al., 1999). However, the level of CAR was not measured.

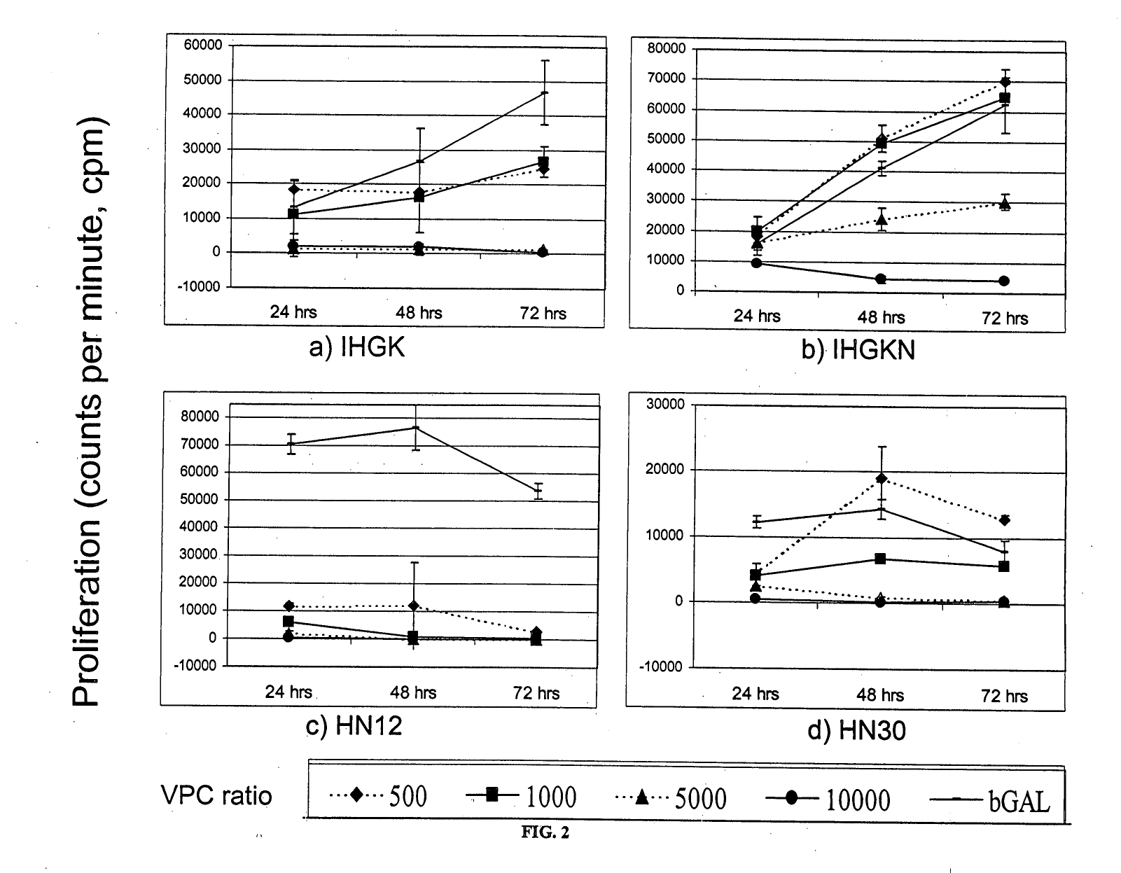

Inhibition of Proliferation. After transduction of IHGK, IHGKN, HN30, and HN12 cells with Ad-p53, proliferation (thymidine incorporation) was suppressed with increasing VPC ratios (FIG. 2(a)-(d)) as compared with controls (Ad-βgal) in all cell lines. HPV-immortalized keratinocytes (IHGK) were more sensitive to p53 suppression than carcinogen transformed cells (IHGKN). When endogenous p53 is inactivated by E6 in the upper aerodigestive tract keratinocytes (IHGK), these cells are susceptible to the effects of exogenous p53. Furthermore, exogenous p53 expression suppressed proliferation in IHGNK cells which have been transformed with a carcinogen. HN12 cells were extremely sensitive to the growth suppressive effects of Ad-p53; transduction with a VPC as low as 500 resulted in a significant inhibition of proliferation when compared with Ad-βgal transduction. Of note, HN12 cells (mutated p53 gene) were more sensitive to p53 suppression at 72 hours than HN30 cells, particularly at lower VPC ratios. The rate of proliferation was inhibited in HN30 (p53 wild-type) at 24 hours (approximately 60% growth suppression relative to Ad-βgal-transduced cells) but increased by 72 hours at lower VPC ratios (<1000), indicating a transient suppression of growth at lower multiplicities of infection in this cell line. Proliferation was suppressed throughout the assay at higher VPC ratios. The results indicate that the sensitivity of HNSCC cells to the antiproliferative effects of Ad-p53 may vary at lower multiplicities of infection but is more consistent at higher multiplicities of infection (>1000 viral particles/cell).

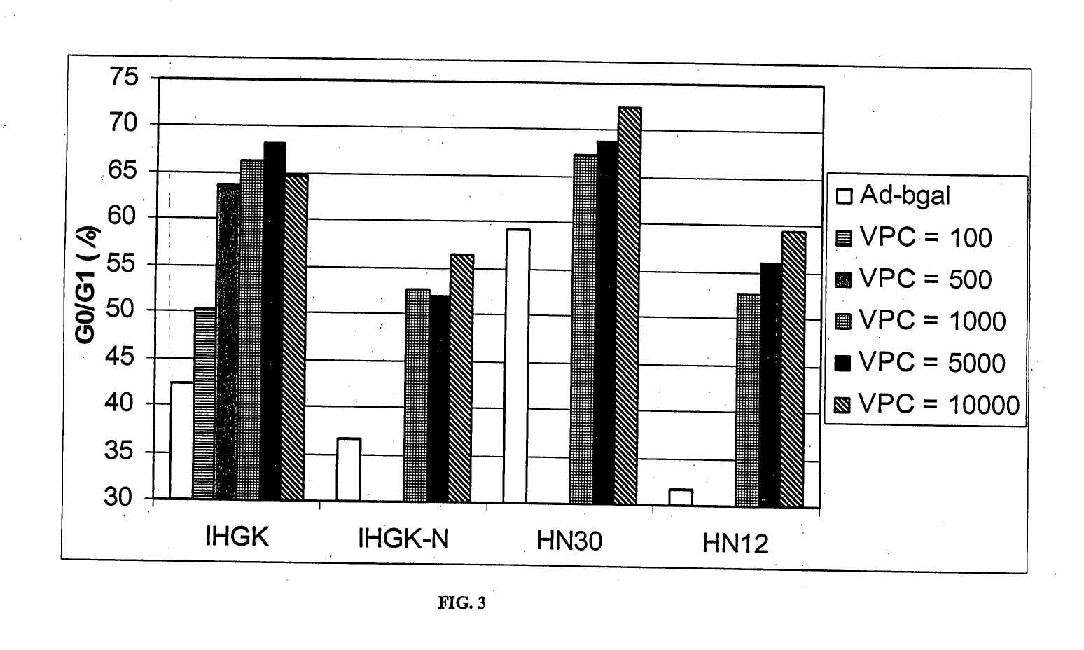

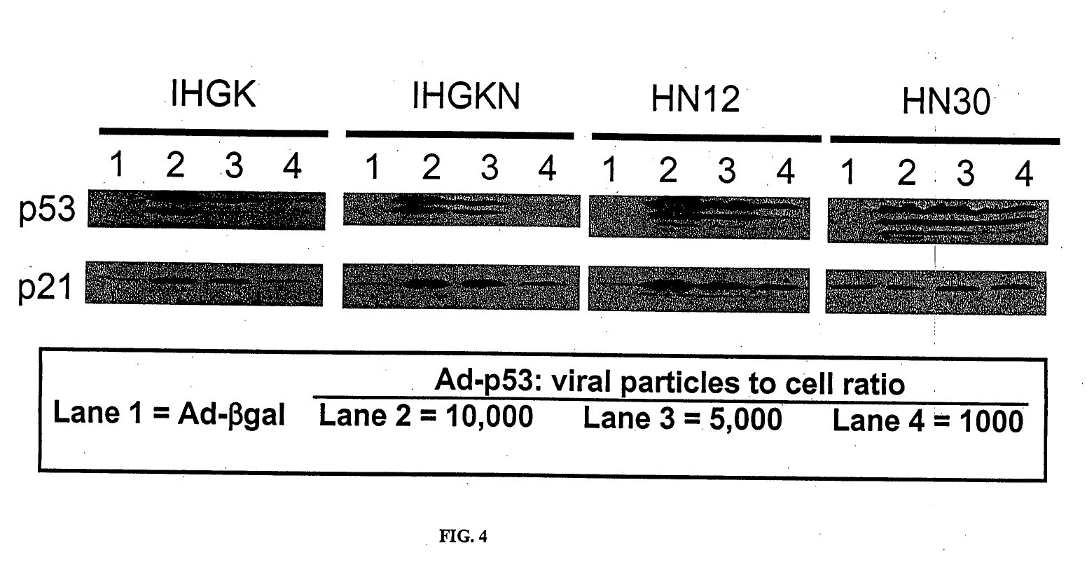

G1 Cell Cycle Arrest. All cell lines were susceptible to p53-induced G1 cell cycle arrest with increasing VPC ratios at 48 hours (FIG. 3). IHGK cells were more sensitive to p53-induced cell cycle arrest than IHGKN cells. Similarly, the increase in p53-induced cell cycle arrest was greater in HN12 than HN30 cells. Most of the HN30 cells (59%) were found in this phase of the cell cycle in response to Ad-gal with an increase to 67% in response to addition of Ad-p53 at a VPC of 1000. The difference in cell cycle distribution between these two HNSCC cell lines both untransduced and in response to Ad-p53 transduction may reflect the p53 status of the cells (HN12 is mutated; HN30 is wild type (Yeudall et al., 1997)). The expression of p53 increased with increasing Ad-p53 VPC ratios (FIG. 4). The expression of p21 was induced in all cell lines except HN30 at VPC ratios of 1000, 5000, and 10,000. No induction of p21 was observed in HN30 although expression ofp53 increased with increasing VPC. HN30 had the highest level of p21 in the Ad-gal transduced cells. Overexpression of p53 without induction of p21 in HN30 may be because of the time period in which these experiments were performed. HN30 cells were examined at 48 hours, and maximum growth suppression occurs at 24 hours (FIG. 2).

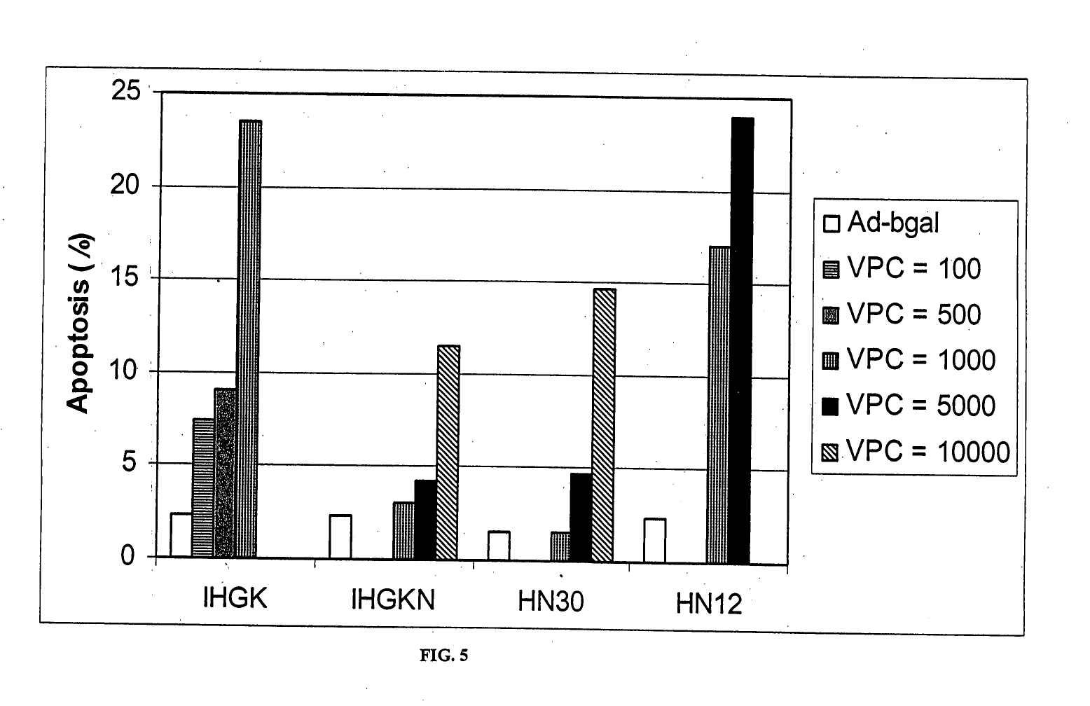

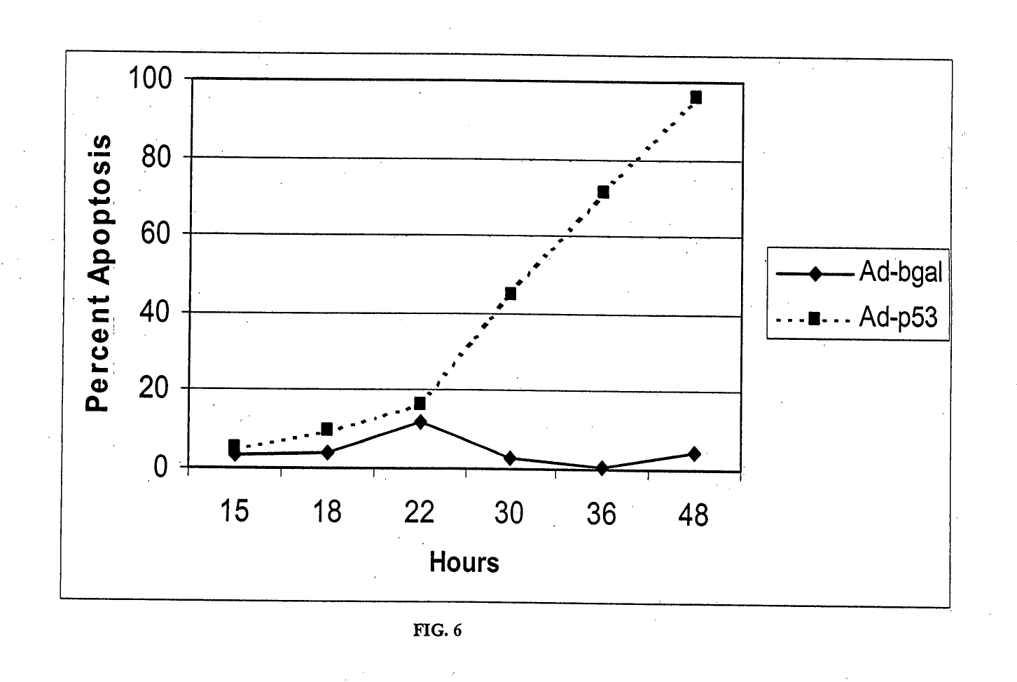

Induction of Apoptosis. As the VPC ratio increased, apoptosis (% annexin binding) also increased in all cell lines at 48 hours in response to Ad-p53 transduction (FIG. 5). IHGK cells were more sensitive to apoptosis induced by Ad-p53 transduction than were carcinogen transformed IHGKN cells. HN12 were the most sensitive to apoptosis induced in response to the vector. At 48 hours, no viable HN12 cells were obtained at a VPC ratio of 10,000. Therefore, a second experiment was conducted with this cell line to determine the kinetics of apoptosis. The level of apoptosis was measured between 15 and 48 hours (FIG. 6). After 22 hours, there was a sharp increase in the rate of apoptosis in HN12 cells. Cell death was linear between 22 and 48 hours with essentially total cell death by 48 hours. HN30 cells (wild-type p53) underwent a dose-dependent increase in apoptosis in response to Ad-p53 transduction, which reached a maximum at 48 hours at a VPC ratio of 10,000 (FIG. 5), similar to the level found in IHGKN cells in response to Ad-p53 transduction. HN30 cells have been show to be resistant to cisplatinum-induced cytotoxicity (Kim et al, 2000).

These results indicate that cell cycle regulation by gene transfer is feasible in immortalized oral keratinocytes. Carcinogen transformed cells are less susceptible to the effects of p53 overexpression. Expression of exogenous p53 through p53 gene transfer can suppress HPV immortalization and carcinogen transformation in oral keratinocytes. The sensitivity of HNSCC cell lines to p53-induced cell cycle regulation and apoptosis is variable and dependent on the cell line and duration of exposure.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### H. REFERENCES

The following references, to the extent that they provide exemplary procedural or other details supplementary to those set forth herein, are specifically incorporated herein by reference.
U.S. Pat. No. 4,554,101
U.S. Pat. No. 4,683,202
U.S. Pat. No. 4,797,368
U.S. Pat. No. 5,139,941
U.S. Pat. No. 5,672,344
U.S. Pat. No. 5,925,565
U.S. Pat. No. 5,928,906
U.S. Pat. No. 5,935,819
Aksentijevich et al., Hum. Gene Ther., 7(9):1111-1122, 1996.
Almendro et al., J. Immunol., 157(12):5411-5421, 1996.
Angel et al., Mol. Cell. Biol., 7:2256, 1987.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Banerji et al., Cell, 27(2 Pt 1):299-308, 1981.
Banerji et al., Cell, 33(3):729-740, 1983.
Batterson and Roizman, J. Virol., 46(2):371-377, 1983.
Beckter et al., Obstet. Gynecol. Clin., 987: 14:389-396, 1987.
Bedi et al., Cancer Res., 56:2484-2487-1996.
Benvenisty and Neshif, Proc. Natl. Acad. Sci. USA, 83(24):9551-9555, 1986.
Berkhout et al., Cell, 59:273-282, 1989.
Bier-Laning et al., Proc. An. Soc. Clin. Oncol., 18:431 a, 1999.
Blanar et al., EMBO J, 8:1139, 1989.
Bodine and Ley, EMBO J, 6:2997, 1987.
Bosch et al., J. Clin. Pathol., 55(4):244-265, 2002.
Boshartetal., Cell, 41:521, 1985.
Bosze et al., EMBO J, 5(7):1615-1623, 1986.
Bousssif et al., Proc. Natl. Acad. Sci. USA, 92(16):7297-7301, 1995.
Boyle et al., Cancer Res., 53:4477-4480, 1993.
Braddock et al., Cell, 58:269, 1989.
Bulla and Siddiqui, J. Virol., 62:1437, 1986.
Caley et al., J. Virology, 71(4):3031-3038, 1997.
Califano et al., Cancer Res., 56:2488-2492, 1996.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Casey et al., Oncogene, 6(10):1791-1797, 1991.
Celander and Haseltine, J. Virology, 61:269, 1987.
Celander et al., J. Virology, 62:1314, 1988.
Chandler et al, Cell, 33:489, 1983.
Chandler et al, Proc. Natl. Acad. Sci. USA, 94(8):3596-601, 1997.
Chang et al., Hepatology, 14:134A, 1991.
Chang et al., Mol. Cell. Biol., 9:2153, 1989.
Chatterjee et al., Proc Natl. Acad. Sci. U.S.A., 86:9114, 1989.
Chen and Okayama, Mol. Cell Biol., 7(8):2745-2752, 1987.
Choi et al., Cell, 53:519, 1988.
Chomchai et al., Laryngoscope, 109:455-459, 1999.
Clark et al., Hum. Gene Ther., 6(10):1329-1341, 1995.
Clayman et al. J. Clin. Oncol., 16:2221-2232, 1998.
Clayman et al., Cancer Res., 55(1): 1-6, 1995.
Clayman et al., Clin. Cancer Res., 5:1715-1722, 1999.
Cocea, Biotechniques, 23(5):814-816, 1997.
Coffin, Retroviridae and Their Replication. In: Virology, Fields et al., eds., Raven Press, New York, pp. 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Cripe et al., EMBO J, 6:3745, 1987.
Cullen et al., J. Virol., 65(2):606-612, 1991.
Culotta and Hamer, Mol. Cell. Biol., 9:1376, 1989.
Dandolo et al., J. Virology, 47:55-64, 1983.
Davis et al., J. Virol., 70(6):3781-3787, 1996.
De Villiers et al., Nature, 312(5991):242-246, 1984.
DeLuca et al., J. Virol., 56(2):558-570, 1985.
Demeret et al., J. Virol., 68(1):7075-7082, 1994.
Denissenko et al., Science, 274:430-432, 1996.
Deschamps et al., Science, 230:1174-1177, 1985.
Dubensky et al., Proc. Natl. Acad. Sci. USA, 81:7529-7533, 1984.
Durstet al., J. Gen. Virol., 66:1515-1522, 1985.
Edbrooke et al., Mol. Cell. Biol., 9:1908, 1989.
Edlund et al., Science, 230:912-916, 1985.
Eicher et al., Clin. Cancer Res., 2(10):1659-1664, 1996.
Elroy-Stein et al., Proc. Natl. Acad. Sci. USA, 86(16):6126-30, 1989.
Fearon and Vogelstein, Cell, 61:759-767, 1990.
Fechheimer et al., Proc. Natl. Acad. Sci. USA, 84:8463-8467, 1987.
Felgner et al., Proc. Natl. Acad. Sci. USA, 84(21):7413-7417, 1987.
Feng and Holland, Nature, 334:6178, 1988.
Firak and Subramanian, Mol. Cell. Biol., 6:3667, 1986.
Flotte et al., Am. J. Respir. Cell Mol. Biol., 7(3):349-356, 1992.
Flotte et al., Proc. Natl. Acad. Sci. USA, 90(22):10613-10617, 1993.
Foecking and Hofstetter, Gene, 45(1):101-105, 1986.
Fraley et al., Proc. Natl. Acad. Sci. USA, 76:3348-3352, 1979.
Fujita et al., Cell, 49:357, 1987.
Furumoto and Irahara, J. Med. Invest., 49(3-4):124-133, 2002.
Gabizon et al., Cancer Res, 50(19):6371-8, 1996.
Ghosh and Bachhawat, In: Liver Diseases, Targeted Diagnosis and Therapy Using Specific Receptors and Ligands. Wu et al. (eds.), Marcel Dekker, NY, 87-104, 1991.
Ghosh-Choudhury et al., EMBO J, 6:1733-1739, 1987.
Gilles et al., Cell, 33:717, 1983.
Gillison et al., J. Natl. Cancer Inst., 92:709-720, 2000.
Glorioso et al., Mol. Biotechnol., 4(1):87-99, 1995.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodboum and Maniatis, Proc. Natl. Acad. Sci. USA, 85:1447, 1988.
Goodboum et al., Cell, 45:601, 1986.
Gopal, Mol. Cell Biol., 5:1188-1190, 1985.
Graham and Prevec, In: Methods in Molecular Biology: Gene Transfer and Expression Protocol, Murray (ed.), Humana Press, Clifton, N.J., 7:109-128, 1991.
Graham and Van Der Eb, Virology, 52:456-467, 1973.
Graham et al., J. Gen. Virl., 36(1):59-74, 1977.
Greene et al., Immunology Today, 10:272, 1989
Grosschedl and Baltimore, Cell, 41:885, 1985.
Grunhaus et al., Seminar in Virology, 200(2):535-546, 1992.
Hamada et al., Cancer Res., 56(13):3047-3054, 1996.
Hamada et al., Gynecol. Oncol., 1996, 60:373-379 and 63:219-227, 1996.
Harland and Weintraub, J. Cell Biol., 101:1094-1099, 1985.
Haslinger and Karin, Proc. Natl. Ac ad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Natl. Acad. Sci. USA, 90:2812-2816, 1993.
Hirochika et al., J. Virol., 61:2599, 1987.
Hirsch et al., Mol. Cell. Biol., 10:1959, 1990.
Holbrook et al., Virology, 157:211, 1987.
Holland and Holland, J Biol Chem, 255(6):2596-605, 1980.
Honess and Roizman, J Virol., 14(1):8-19, 1974.
Honess and Roizman, Proc. Natl. Acad. Sci. USA, 72(4):1276-1280.1975.
Hong et al., N. Engl. J. Med., 315:1501-1505, 1986.
Hong et al., N. Engl. J. Med., 323:795-801, 1990.
Hopp et al., BioTechnology, 7:1205-1210, 1988.
Horlick and Benfield, Mol. Cell. Biol., 9:2396, 1989.
Horwich et al., Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawaetal., Cell, 51:251, 1987.
Imbra and Karin, Nature, 323:555, 1986.
Imler et al., Mol. Cell. Biol, 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jastreboff and Cymet, Postgrad. Med. J., 78(918):225-228, 2002.
Jaynes et al., Mol. Cell. Biol., 8:62, 1988.
Jeon et al., J. Virol., 69(5):2989-2997, 1995.
Johnson et al., Mol. Cell. Biol., 9:3393, 1989.
Jones and Shenk, Cell, 13:181-188, 1978.
Kadesch and Berg, Mol Cell. Biol., 6:2593, 1986.
Kaneda et al., Science, 243:375-378, 1989.
Kaplitt et al., Nat Genet., 8(2):148-54, 1994.
Karin et al., Mol. Cell. Biol., 7:606, 1987.
Karlsson et al., EMBO J., 5:2377-2385, 1986.
Katinka et al., Cell, 20:393, 1980.
Kato et al, J. Biol. Chem., 266:3361-3364, 1991.
Kawamoto et al., Mol. Cell. Biol., 8:267, 1988.
Khuri et al., J. Natl. Cancer Inst., 89:199-211, 1997.
Kiledjian et al., Mol. Cell. Biol., 8:145, 1988.
Kim et al, Clin. Cancer Res., 6:4142-4147, 2000.
Klamut et al., Mol. Cell. Biol., 10:193, 1990.
Klein et al., Nature, 327:70-73, 1987.
Koch et al., J. Natl. Cancer Inst., 88:1580-1586, 1996.
Koch et al., Mol. Cell. Biol., 9:303, 1989.
Kotin et al., Proc. Natl. Acad. Sci. USA, 87(6):2211-5, 1990.
Kraus et al. FEBS Lett., 428(3):165-170, 1998.
Kriegler and Botchan, In: Eukaryotic Viral Vectors, Gluzman (ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaFace et al., Virology, 162(2):483-486, 1988.
Lareyre et al., J. Biol. Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Le Gal La Salle et al., Science, 259:988-990, 1993.
Lebkowski et al., Mol. Cell. Biol., 8(10):3988-3996, 1988.
Lee et al., Biochem. Biophys. Res. Commun., 240(2):309-13, 1997.
Lee et al., Nature, 294:228, 1981.
Lee et al., Nucleic Acids Res., 12:4191-206, 1984.
Levenson et al., Hum Gene Ther. 20;9(8):1233-1236, 1998.
Levinson et al., Nature, 295:79, 1982.
Levrero et al., Gene, 101: 195-202, 1991.
Li et al., Clin. Cancer Res., 5:4175-81, 1997.
Lin et al., Mol. Cell. Biol., 10:850, 1990.
Liuetal., Cancer Res., 55:3117-3122, 1995.
Luo et al., Proc. Natl. Acad. Sci. USA, 93:8907-8912, 1996.
Luria et al., EMBO J., 6:3307, 1987.
Lusky and Botchan, Proc Natl. Acad. Sci. U.S.A., 83:3609, 1986.
Lusky et al., Mol. Cell. Biol. 3:1108, 1983.
Lydiatt et al., Cancer, 82:1376-1380, 1998.
Macejak and Samow, Nature, 353:90-94, 1991.
Majors and Varmus, Proc. Natl. Acad. Sci. USA, 80:5866, 1983.
Mann et al., Cell, 33:153-159, 1983.
Mao et al., Nat. Med., 2:682-685, 1996.
Markowitz et al., J. Virol., 62:1120-1124, 1988.
Matsukura et al., Virology, 172(1):63-72, 1989.
McCarty et al., J. Virol., 65(6):2936-45, 1991.
McLaughlin et al., J. Virol., 62(6):1963-1973, 1988.
McNeall et al., Gene, 76:81, 1989.
Mercer, Crit. Rev. Eukaryot. Gene Expr., 2(3):251-63, 1992.
Miksicek et al., Cell, 46:203, 1986.
Montenarh, Oncogene, 7(9):1673-80, 1992.
Mordacq and Linzer, Genes and Dev., 3:760, 1989.
Moreau et al., Nucl. Acids Res., 9:6047, 1981.
Muesing et al., Cell, 48:691, 1987.
Munck-Wikland et al., Head Neck, 19:107-115, 1997.
Muzyczka, Curr. Topics Microbiol. Immunol., 158:97-129, 1992.
Ng et al., Nuc. Acids Res., 17:601, 1989.
Nicolas and Rubenstein, In: Vectors: A survey of molecular cloning vectors and their uses, Rodriguez and Denhardt (eds.), Stoneham: Butterworth, 493-513, 1988.
Nicolau and Sene, Biochim. Biophys. Acta, 721:185-190, 1982.
Nishizaki et al., Clin. Cancer Res., 5:1015-1023, 1999.
Nomoto et al., Gene, 236(2):259-271, 1999.
Oda et al., Carcinogenesis, 17:2003-2008, 1996.
Ohi et al., Gene, 89(2):279-282, 1990.
Ondek et al., EMBO J., 6:1017, 1987.
Ornitz et al., Mol. Cell. Biol., 7:3466, 1987.
Palmiter et al., Nature, 300:611, 1982.
Papadimitrakopoulou et al., Oncogene, 14:1799-1803, 1997.
Paskind et al., Virology, 67:242-248, 1975.
PCT App. WO 98/07408
Pech et al., Mol. Cell. Biol., 9:396, 1989.
Pelletier and Sonenberg, Nature, 334:320-325, 1988.
Perez-Stable and Constantini, Mol. Cell. Biol., 10: 1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Post et al., Cell, 24(2):555-65, 1981.
Potteretal., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Riccioni et al., Gene Ther., 5:747-754, 1998.
Rich et al., Hum. Gene Ther., 4:461-476, 1993.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sakai et al., Genes and Dev., 2:1144, 1988.
Salzman and Howley, In: The papovaviridae, Plenum Press, NY, 2:199-243, 1987.
Sambrook et al., In: Molecular cloning, Cold Spring Harbor Laboratory Press, Cold Spring Harbor, N.Y., 2001.
Samulski et al., J. Virol, 63:3822-3828, 1989.
Satake et al., J. Virology, 62:970, 1988.
Sauter et al., Clin. Cancer Res., 5:775-782, 1999.
Schaffner et al., J. Mol. Biol., 201:81, 1988.
Schneider-Gadicke et al. EMBO J., 5:2285-2292, 1986.
Schuller et al., Pharmacol. Ther., 46:95-103, 1990.
Schwarz, et al., Nature, 314:111-114, 1985.
Searleetal., Mol. Cell. Biol., 5:1480, 1985.
Sharp and Marciniak, Cell, 59:229, 1989.
Shaul and Ben-Levy, EMBO J., 6:1913, 1987.
Shelling and Smith, Gene Therapy, 1: 165-169, 1994.
Sherman et al., Mol. Cell. Biol., 9:50, 1989.
Sisk et al., Head Neck, 24(9):841-849, 2002.
Sleigh and Lockett, J. EMBO, 4:3831, 1985.
Smith and Moss, Gene, 25(1):21-8, 1983.
Solodin et al., Biochemistry, 34(41):13537-13544, 1995.
Spalholzetal., Cell, 42:183, 1985.
Spandau and Lee, J. Virology, 62:427, 1988.
Spandidos and Wilkie, EMBO J, 2:1193, 1983.
Steinberg and DiLorenzo, Cancer Metastasis Rev.,, 15:91-112, 1996.
Stephens and Hentschel, Biochem. J, 248:1, 1987.
Stratford-Perricaudet and Perricaudet, In: Human Gene Transfer, Cohen-Haguenauer and
Boiron (eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takahasi et al., In Vitro Cell Dev Biol,. 28A(6):380-2, 1992.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (ed.), New York: Plenum Press, pp. 149-188, 1986.
Thierry et al., Proc Natl Acad Sci USA. 92(21):9742-9746, 1995.
Thiesen et al., J. Virology, 62:614, 1988.
Top et al., J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
Treisman, Cell, 42:889, 1985.
Tronche et al., Mol. Biol. Med., 7:173, 1990.
Trudel and Constantini, Genes and Dev. 6:954, 1987.
Tsukamoto et al., Nat. Genet., 9(3):243-248, 1995.
Tsumaki et al., J. Biol. Chem., 273(36):22861-22864, 1998.
Turek, Adv Virus Res., 44:305-356, 1994.
Tur-Kaspa et al., Mol. Cell Biol., 6:716-718, 1986.
Tyndell et al., Nuc. Acids. Res., 9:6231, 1981.
Ushikai et al., J. Virol., 68(1):6655-6666, 1994.
Vannice and Levinson, J. Virology, 62:1305, 1988.
Vasseur et al., Proc Natl. Acad. Sci. U.S.A., 77:1068, 1980.
Wagner et al., Science, 260:1510-1513, 1993.
Walsh et al., J. Clin. Invest, 94:1440-1448, 1994.
Wang and Calame, Cell, 47:241, 1986.
Watanabe et al., Exper. Cell Res., 230:76-83, 1997.
Weber et al., Cell, 36:983, 1984.
Wei et al., Cancer Res., 56:3975-3979, 1996.
Wei et al., Gene Therapy, 1:261-268, 1994.
Weinberg, Science, 254(5035):1138-1146, 1991.
Weinberger et al. Mol. Cell. Biol., 8:988, 1984.
Wilczynski et al., Virology, 166:624-267, 1988.
Winoto and Baltimore, Cell 59:649, 1989.
Wong et al., Gene, 10:87-94, 1980.
Wu and Wu, Biochemistry, 27:887-892, 1988.
Wu and Wu, J. Biol. Chem., 262:4429-4432, 1987.
Wu et al., Biochem. Biophys. Res. Commun., 233(1):221-6, 1997.
Yang and Huang, Gene Therapy, 4 (9):950-960, 1997.
Yang et al., J. Virol., 68:4847-4856, 1994.
Yang et al., Proc Natl. Acad. Sci. USA, 87:9568-9572, 1990.
Yeudall et al., Carcinogenesis, 15:2683-2686, 1994.
Yeudall et al., Mol. Carcinog., 18:89-96, 1997.
Yoder et al., Blood, 82 (Supp.): 1:347A, 1994.
Yoo et al., Arch. Otolaryngol. Head Neck Surg., 126:1313-1318, 2000.
Yutzeyetal. Mol. Cell. Biol., 9:1397, 1989.
Zhao-Emonet et al., Biochim Biophys Acta, 1442(2-3):109-119, 1998.
Zhou et al., Exp. Hematol, 21:928-933, 1993.
Zhou et al., J. Exp. Med., 179:1867-1875, 1994.
Zhu et al., Science, 261(5118):209-211, 1993.

1. A method of inhibiting the growth of a papillomavirus-transformed cell in a hyperplastic lesion in a subject comprising topically administering to said lesion a growth inhibiting amount of a composition comprising (a) an expression cassette comprising a promoter, active in cells of said lesion, operably linked to a polynucleotide encoding a p53 polypeptide, and (b) a pharmaceutical preparation suitable for topical delivery, wherein expression of said p53 polypeptide inhibits growth of said cell.

2. The method of claim 1, wherein said subject is a mammal.

3. The method of claim 2, wherein said mammal is a human.

4. The method of claim 1, wherein said cell is a keratinocyte.

5. The method of claim 1, wherein said cell is an epithelial cell.

6. The method of claim 1, wherein said cell is a skin cell.

7. The method of claim 1, wherein said cell is a mucosal cell.

8. The method of claim 1, wherein said papillomavirus is a human papillomavirus.

9. The method of claim 1, wherein said lesion is selected from the group consisting of a squamous cell hyperplastic lesion, premalignant epithelial lesion, psoriatic lesion, cutaneous wart, periungual wart, anogenital wart, epidermodysplasia verruciformis, an intraepithelial neoplastic lesion, focal epithelial hyperplasia, conjunctival papilloma, conjunctival carcinoma, or squamous carcinoma lesion.

10. The method of claim 1, wherein said expression cassette is carried in a viral vector.

11. The method of claim 10, wherein said viral vector is an adenoviral vector, a retroviral vector, an adeno-associated viral vector, a vaccinia viral vector or a pox viral vector.

12. The method of claim 10, wherein said viral vector is an adenoviral vector.

13. The method of claim 1, wherein said expression cassette is carried in a nonviral vector.

14. The method of claim 13, wherein said non-viral vector is a lipid.

15. The method of claim 1, wherein said composition is formulated as a mouthwash or mouthrinse.

16. The method of claim 15, wherein said mouthwash further comprises a flavorant.

17. The method of claim 16, wherein said flavorant is selected from the group comprising one or more flavor components selected from wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, cinnamon leaf oil, and mixtures thereof.

18. The method of claim 1, wherein said composition is formulated as a douche solution.

19. The method of claim 1, wherein said composition is formulated as an ointment or salve.

20. The method of claim 1, wherein said composition is formulated as a cream for topical, anal or vaginal delivery.

21. The method of claim 1, wherein said composition is formulated as a spray or aerosol.

22. The method of claim 1, wherein said composition is formulated as a suppository for anal or vaginal delivery.

23. The method of claim 1, wherein the promoter is a constitutive promoter, an inducible promoter or a tissue specific promoter.

24. The method of claim 1, wherein inhibiting growth comprises in slowing or halting the growth of said lesion.

25. The method of claim 1, wherein inhibiting growth comprises a reduction in the size of said lesion.

26. The method of claim 1, wherein inhibiting growth comprises induction of apoptosis said cells of said lesion.

27. The method of claim 1, wherein inhibiting growth comprises induction of an immune response against said cells of said lesion.

28. The method of claim 1, further comprising subjecting said subject to a secondary anti-hyperplastic therapy.

29. The method of claim 28, wherein said secondary anti-hyperplastic therapy is chemotherapy, radiotherapy, immunotherapy, phototherapy, cryotherapy, toxin therapy, hormonal therapy or surgery.

30. A mouthwash for inhibiting the growth of a papillomavirus-transformed cell in a hyperplastic lesion in a subject comprising (a) an expression cassette comprising a promoter operably linked to a polynucleotide encoding a p53 polypeptide, and (b) a liquid carrier formulated for oral delivery.

31. The mouthwash of claim 30, further comprising a flavorant.

32. The mouthwash of claim 31, wherein said flavorant is selected from the group comprising one or more flavor components selected from wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, cinnamon leaf oil, and mixtures thereof.

33. A douche solution for inhibiting the growth of a papillomavirus-transformed cell in a hyperplastic lesion in a subject comprising (a) an expression cassette comprising a promoter operably linked to a polynucleotide encoding a p53 polypeptide, and (b) a liquid carrier formulated for vaginal delivery.

34. A suppository for inhibiting the growth of a papillomavirus-transformed cell in a hyperplastic lesion in a subject containing (a) an expression cassette comprising a promoter operably linked to a polynucleotide encoding a p53 polypeptide, and (b) formulated for anal or vaginal delivery.

35. A cream for inhibiting the growth of a papillomavirus-transformed cell in a hyperplastic lesion in a subject containing (a) an expression cassette comprising a promoter operably linked to a polynucleotide encoding a p53 polypeptide, and (b) formulated for topical, anal or vaginal delivery.

36. A solution for inhibiting the growth of a papillomavirus-transformed cell in a hyperplastic lesion in a subject containing (a) an expression cassette comprising a promoter operably linked to a polynucleotide encoding a p53 polypeptide, and (b) formulated for hypospray.

37. An aerosolized suspension for inhibiting the growth of a papillomavirus-transformed cell in a hyperplastic lesion in a subject containing an expression cassette comprising a promoter operably linked to a polynucleotide encoding a p53 polypeptide.

38. A method of suppressing or preventing papillomavirus-mediated transformation of a cell in a subject comprising administering to said cell a composition comprising (a) an expression cassette comprising a promoter, active in said cell operably linked to a polynucleotide encoding a p53 polypeptide, and (b) a pharmaceutical preparation suitable for topical delivery, wherein expression of said p53 suppresses said transformation.

39. The method of claim 38, wherein said cell is a keratinocyte.

40. The method of claim 38, wherein said subject is a human.

41. The method of claim 38, wherein said subject is a human at risk of developing an oral hyperplastic lesion.

42. The method of claim 41, wherein said human at risk of developing an oral hyperplastic lesion is a human with a history of a previous oral hyperplastic lesion.

43. The method of claim 42, wherein said previous oral hyperplastic lesion is comprised of cells selected from the group consisting of premalignant epithelial cells, squamous intraepithelial neoplastic cells, squamous hyperplastic cells, and squamous cell carcinoma cells.

44. The method of claim 41, wherein said oral hyperplastic lesion is comprised of cells transformed by a papillomavirus.

45. The method of claim 44, wherein said papillomavirus is a human papillomavirus.

46. The method of claim 38, wherein said expression cassette is carried in a viral vector.

47. The method of claim 46, wherein said viral vector is an adenoviral vector, a retroviral vector, an adeno-associated viral vector, a vaccinia viral vector or a pox viral vector.

48. The method of claim 46, wherein said viral vector is an adenoviral vector.

49. The method of claim 38, wherein said expression cassette is carried in a nonviral vector.

50. The method of claim 49, wherein said nonviral vector is a lipid.

51. The method of claim 38, wherein said composition is formulated as a mouthwash or mouth rinse.

52. The mouthwash of claim 51, further comprising a flavorant.

53. The mouthwash of of claim 52, wherein said flavorant is selected from the group comprising one or more flavor components selected from wintergreen oil, oregano oil, bay leaf oil, peppermint oil, spearmint oil, clove oil, sage oil, sassafras oil, lemon oil, orange oil, anise oil, benzaldehyde, bitter almond oil, camphor, cedar leaf oil, marjoram oil, citronella oil, lavendar oil, mustard oil, pine oil, pine needle oil, rosemary oil, thyme oil, cinnamon leaf oil, and mixtures thereof.

54. The method of claim 38, wherein said composition is formulated as a douche solution for vaginal delivery.

55. The method of claim 38, wherein said composition is formulated as a suppository for anal or vaginal delivery.

56. The method of claim 38, wherein said composition is formulated as an ointment or salve.

57. The method of claim 38, wherein said composition is formulated as a cream for topical, anal or vaginal delivery.

58. The method of claim 38, wherein said composition is formulated as a spray or aerosol.

59. The method of claim 38, wherein said composition is formulated as a pill or capsule.

60. The method of claim 38, wherein said composition is formulated for timed-release.
